(19) United States
(12) Patent Application Publication
(10) Pub. No.: US 2009/0043159 A1
(43) Pub. Date: Feb. 12, 2009

(54) SLEEVE FOR ENDOSCOPIC MEDICAL PROCEDURES

(75) Inventor: John Dumot, Chagrin Falls, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/053,007

(22) Filed: Mar. 21, 2008

Related U.S. Application Data
(60) Provisional application No. 60/920,416, filed on Mar. 28, 2007.

(51) Int. Cl. A61B 1/01

(52) U.S. Cl. 600/114

(57) ABSTRACT

A guide tube is used with a medical endoscope. The guide tube includes an elongated tubular member and a lubricating fluid. The elongated tubular member has an inner annular membrane, an outer annular membrane, a proximal end portion, and a distal end portion opposite the proximal end portion. The inner and outer annular membranes define a longitudinal axis extending between the proximal end portion and the distal end portion. The lubricating fluid is disposed between the inner annular membrane and the outer annular membrane. The lubricating fluid is disposed between the distal end portion and the proximal end portion. The inner annular membrane moves in a first axial direction along the medical endoscope as the medical endoscope is moved through a patient. The outer annular membrane correspondingly moving axially, but in a second, opposite axial direction, along the medical endoscope as the medical endoscope is moved through the patient.

### RELATED APPLICATION

This application claims priority from U.S. Provisional Application No. 60/920,416, filed Mar. 28, 2007. The subject matter of the aforementioned application is incorporated herein by reference in its entirety.

### FIELD OF INVENTION

The field of the present invention relates generally to a device for use in medical examinations, and more specifically, to a device for use in endoscopic examinations of the human colon.

### BACKGROUND OF THE INVENTION

Colonoscopy is typically the most sensitive and specific means for examining the colon, particularly for the diagnosis of colon cancers and polyps. Because the cecum, the portion of the colon furthest from the anus, is a common location for cancer, it is important that the entire colon be completely examined. In addition, cancers of the colon have become more common. However, because the anatomy of the colon can vary from person to person, the technique of a total colonoscopy is technically demanding.

During a colonoscopy, the colonoscope is inserted in the anus, through the rectum, sigmoid colon, descending colon, transverse colon, ascending colon, and into the cecum. Advancing the colonoscope, typically 160 centimeters in length, may be difficult due to a loop, or bend, defined by the sigmoid colon. Once the colonoscope reaches the descending colon or transverse colon, this loop is reduced by withdrawing the colonoscope to a straightened position. Failure to straighten the loop in the sigmoid colon prior to continuing may cause enlargement of the loop and result in pain and damage, including perforations and adverse cardiac reactions such as hypotension and bradycardia. Once the loop is straightened, further advancement of the colonoscope can usually be accomplished. Adhesions from prior perforations or surgical procedures can cause a fixed sigmoid colon, which will prevent straightening the sigmoid colon.

In patients having long sigmoid colons, reaching the cecum may be extremely difficult, if not impossible, due to reformation of the sigmoid loop when the operator attempts further advancement of the colonoscope. Continued pushing of the colonoscope under these circumstances enlarges the loop, rather than advancing the tip of the scope. Colonoscope operators often employ various maneuvers to complete the examination, including changing the patient's position and the use of abdominal compressions. Unfortunately, these maneuvers prolong the procedure and are not always successful in helping the scope reach the cecum. Even expert colonoscopists may experience difficulties in 10-20% of cases because of a long sigmoid colon of the patient. The overall success rate for total colonoscopy, i.e. getting the tip of the colonoscope to the cecum, has been reported to be 55-98%.

Accordingly, it is desirable to provide a safer, more effective, and easier to use device for facilitating the insertion and use of a colonoscope during a colonoscopy. It is further desirable to provide a device that reduces friction and imparted stress between the endoscope and the colon during a colonoscopy.

### DESCRIPTION OF THE PRIOR ART

A conventional sigmoid splint has proven to be a useful tool for allowing successful examination of the cecum of a human patient with a colonoscope. The conventional splint is a hollow cylinder about 40 centimeters in length. It is made of a flexible material one millimeter in thickness. The distal end of the conventional splint is tapered to an inner diameter about 4 millimeter greater than the colonoscope. Its proximal end is much larger in diameter in order to prevent entry into the patient's body and has a rubber diaphragm with a hole slightly larger in diameter than the outside diameter of the colonoscope to allow a tight insertion of the scope through the splint.

In use during a colonoscopy, the colonoscope is inserted into the colon until further progress becomes difficult, at which time the tip of the colonoscope is typically located in the upper descending colon or the transverse colon. Using the colonoscope itself, the sigmoid loop of the colon is initially straightened. The conventional splint, placed over the proximal shaft of the colonoscope to begin examination, is gently pushed into the colon over the scope with the aid of lubricants. With full insertion of the splint, the proximal end of the splint remains outside the anus, while the distal end is located in the descending colon. The placement of the splint maintains an almost straight-line passage from the anus to the descending colon. The colonoscope is then advanced while the splint is held stationary. The conventional splint maintains the sigmoid in a straightened position, prevents a loop from reforming, and facilitates advancement of the colonoscope to the cecum.

Despite its advantages, the conventional splint is very rarely utilized due to the risk of perforation of the colon by the splint. Perforations are caused by the entrapment of a piece of colonic wall between the distal end of the splint and the colonoscope when the splint is moved inside the patient's colon. The entrapment may occur either when the splint is advanced over the colonoscope or when the colonoscope is withdrawn through the splint. Once entrapped, a piece of colonic wall may be sheared off by the relatively sharp edge of the distal end of the splint. If the entrapped piece of colonic lining includes all layers of the colonic wall, then the entrapped piece becomes a complete perforation. The conventional splint for the colonoscope may also have useful applications in the upper gastrointestinal tract or other parts of the human anatomy.

Another conventional tubular inserting device has variable rigidity. A long overtube is made of two thin walls with space between. The outer wall is made of semi-rigid, non-expandable material, while the inner wall is made of material which is radially deformable, allowing expansion and contraction of the intervening space. Fluid is injected into the proximal end of the overtube to expand the space. When the space is expanded, the two walls separate from each other creating a flexible overtube. Suctioning out the injected fluid contracts the space, bringing the two walls into contact with each other. The external surface of the inner wall and the internal surface of the outer wall are lined with a cogwheel-like arrangement that engage and lock against each other when the space between them is removed, thereby producing a rigid overtube. When the space is expanded by injecting fluid, the two walls separate and the overtube returns to its flexible state.

This conventional overtube is introduced over the colonoscope. During a colonoscopy, when flexibility of the overtube is desired, the space between the two walls is expanded. When rigidity of the overtube is desired, this space is eliminated. The conventional overtube does not overcome the disadvantage of the space between the distal edge of the splint and the colonoscope entrapping and shearing off of part of the colon wall. Further, the conventional overtube requires the operator to negotiate the sigmoid colon with the endoscope before the overtube is inserted into the patient.

### SUMMARY OF THE INVENTION

A guide tube in accordance with the present invention is used with a medical endoscope. The guide tube includes an elongated tubular member and a lubricating fluid. The elongated tubular member has an inner annular membrane, an outer annular membrane, a proximal end portion, and a distal end portion opposite the proximal end portion. The inner and outer annular membranes define a longitudinal axis extending between the proximal end portion and the distal end portion. The lubricating fluid disposed between said inner annular membrane and said outer annular membrane, said inner annular membrane moving in a first axial direction along the medical endoscope as the medical endoscope is moved through a patient, said outer annular membrane correspondingly moving axially, but in a second, opposite axial direction, along the medical endoscope as the medical endoscope is moved through the patient.

A method in accordance with the present invention is for viewing the interior of a colon of a patient. The method comprises the steps of: providing a lubricating fluid between an inner annular membrane and an outer annular membrane; moving the inner annular membrane in a first axial direction along a medical endoscope as the medical endoscope is moved through a patient; and moving the outer annular membrane correspondingly, but in a second, opposite axial direction, along the medical endoscope as the medical endoscope is moved through the patient.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a schematic representation of a device in accordance with the present invention for examining a human colon;

FIG. 2 is a schematic representation of the device of FIG. 1 in a different position relative to the human colon;

FIG. 3 is a schematic representation of the device of FIG. 1 in a still different position relative to the human colon;

FIG. 4 is a schematic representation of the device of FIG. 1 in a yet different position relative to the human colon;

FIG. 5 is a schematic representation of the device of FIG. 1 in a still different position relative to the human colon;

FIG. 6 is a schematic representation of the device of FIG. 1 in a yet different position relative to the human colon;

FIG. 7 is a schematic representation of the device of FIG. 1 in a still different position relative to the human colon; and

FIG. 8 is a schematic representation of the FIG. 1 in an unused condition.

### DESCRIPTION OF AN EXAMPLE EMBODIMENT

As used herein, the term “sleeve” is used interchangeably with the term “overtube” or “guide tube” to generally describe a device in accordance with the present invention. The term “endoscope” or “scope” is used to refer to a colonoscope, gastroscope, enteroscope, or other type of medical endoscope. In referring to the opposite ends of the scope or sleeve, the “proximal end” means that part of the scope or sleeve which is closest to the operator or physician/surgeon performing a colonoscopy and the “distal end” means that part of the scope or sleeve farthest from the operator or physician/surgeon.

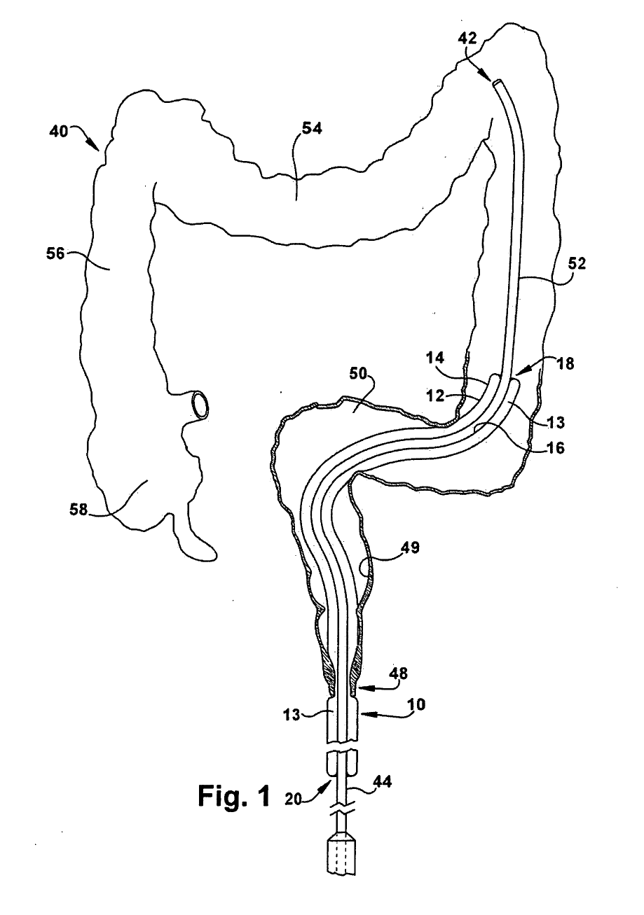

With reference FIGS. 1-8, a guide tube, or sleeve 10, in accordance with one example of the present invention includes an elongated tubular main body 12 with an outer surface 14 adjacent the wall 49 of a human colon 40 and an inner surface 16 adjacent a colonoscope 38. The main body 12 has opposing ends, a distal end 18 which enters the human body first and a proximal end 20 which enters the human body second during the procedures described herein.

The main body 12 comprises a vinyl membrane folded over onto itself and sealed to define a double-walled toroidal structure with a closed interior cavity 13. The main body 12 may be between 30 to 60 centimeters or any suitable length. The closed interior cavity 13 is filled with a lubricating material such as a suitable saline or other nontoxic viscous solution. An instrument, such as the colonoscope 38, may be inserted through the opening defined by the inner surface 16 of the main body 12 of the sleeve 10 (FIG. 8).

Once the colonoscope 38 and sleeve 10 are within the colon 40, no sliding occurs between the colonoscope 38 and the inner surface 16 of the main body 12. Instead, a “rolling” action occurs so that the inner surface 16 folds outward and becomes the outer surface 14 of the main body 12 as the colonoscope 38 is advanced through the colon 40.

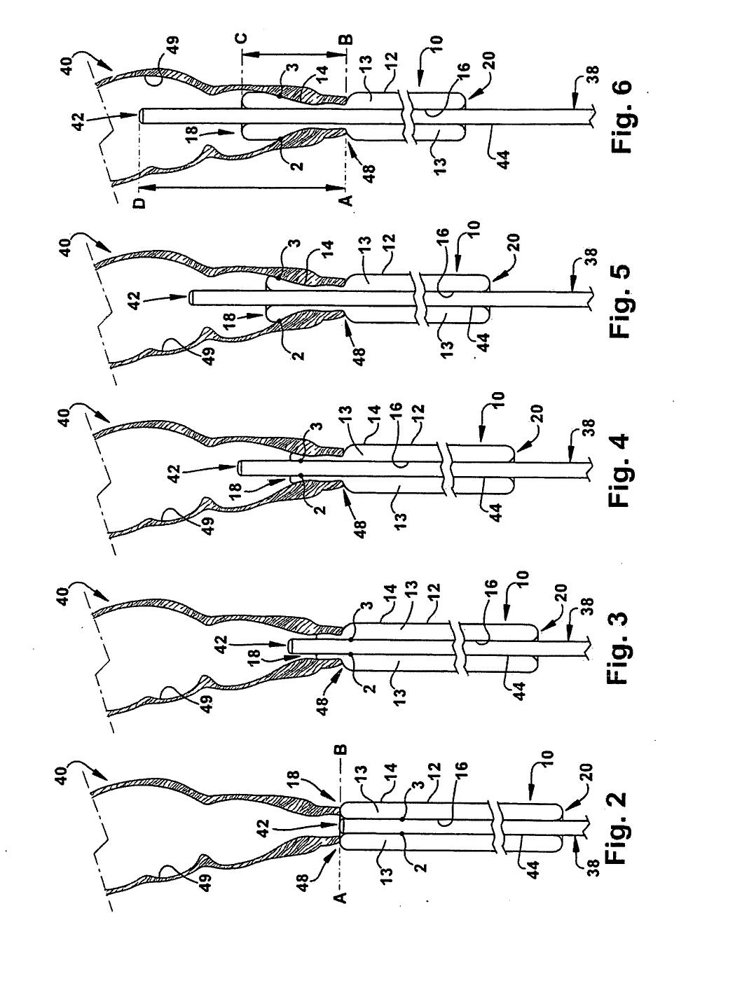

As shown in FIG. 2, the line A-B indicates the distal end 18 of the sleeve 10 and the distal end of the colonoscope 38. Points 2 and 3 indicate two diametrically opposite portions on the cylindrical inner surface 16 of the sleeve 10. As shown in FIG. 3, as the colonoscope 38 and sleeve 10 are inserted in the anus 48, a “rolling” occurs such that the distal end 42 of the colonoscope 38 advances at a rate twice that of the distal end 18 of the sleeve 10. Because of this effect, points 2 and 3 on the inner surface 16 of the sleeve 10 advance at a one half that of the colonoscope 38 and the outer surface 14 of the sleeve 10 does not slide relative to the inner wall 49 of the colon 40.

As shown in FIG. 4, points 2 and 3 approach the distal end 18 of the sleeve 10. As shown in FIGS. 5 and 6, points 2 and 3 have “rolled” over to the outer surface 14 and remain stationary relative to the inner wall 49 of the colon 40 until points 2 and 3 “roll back” to the inner surface 16 of the sleeve 10. Note that the distance B-C between the insertion point at the anus 48 and the distal end 18 of the sleeve 10 is half the distance A-D between the insertion point and the distal end 42 of the colonoscope 38.

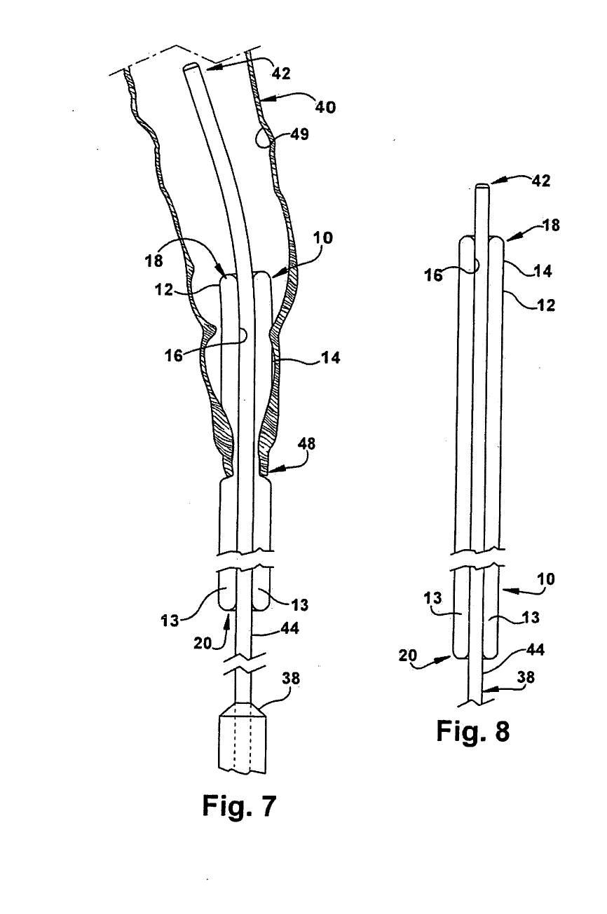

As shown in FIG. 7, the colonoscope 38 and sleeve 10 have completely entered the colon 40. The above described “rolling” thus allows the colonoscope 38 to advances through the colon 40 with little stress and/or pressure transmitted to the inner wall 49 of the colon 40. As the colonoscope 38 is further advanced through colon 40, points 2 and 3 of the sleeve 10 will eventually become part of the inner surface 16 again.

In use during a colonoscopy, as shown in FIGS. 1-8, the tip 42 and body 44 of the colonoscope 38 is initially inserted into the sleeve 10 prior to beginning the procedure to investigate the colon 40 (FIG. 8). The colonoscope 38 and sleeve 10 are introduced into the rectum 46 through the anus 48 and then into the sigmoid colon 50. After negotiating the loop in the sigmoid colon 50, the colonoscope 38 and sleeve 10 enter the descending colon 52. Further advancement of the tip 42 may be made easier by partially withdrawing the colonoscope 38 and sleeve 10 to straighten the sigmoid colon 50 with the body 44 of colonoscope 38. Once the sigmoid colon 50 is straightened, the colonoscope 38 and sleeve 10 are advanced beyond the sigmoid colon 50 toward the descending colon 52. The “rolling action” of the sleeve 10 inside the sigmoid colon 50 and descending colon 52 facilitates further insertion and retraction of the colonoscope 38 through the transverse colon 54 and ascending colon 56 and then into the cecum 58. Force transfer and pressure from the colonoscope 38 to the colon wall 50 is distributed across the fluid filled sleeve 10. Thus, torqueing, pushing, and steering of the colonoscope 38 is reduced as well. As shown in FIGS. 1-8, the “rolling” action of the sleeve 10 provides that the sleeve 10 moves at a rate of insertion of about half that of the tip of the colonoscope 38. After completing the procedure, the sleeve 10 facilitates removal of the colonoscope 38 as well.

Consequently, the “rolling” sleeve 10 with closed lubricated chamber reduces friction between the sigmoid colon 50 and the colonoscope 38 during advancement and retreat of the colonoscope 38. The sleeve 10 may be disposable and sealed such that leakage of the lubricating material does not occur. The sleeve 10 is more flexible than conventional overtubes thereby mitigating trauma to the colon wall 49. Use of the sleeve 10 may overcome previously failed complete insertions.

While there is shown and described herein certain specific alternative forms of the invention, it will be readily apparent to those skilled in the art that the invention is not so limited, but is susceptible to various modifications and rearrangements in design and materials without departing from the spirit and scope of the invention. In particular, it should be noted that the present invention is subject to modification with regard to the dimensional relationships set forth herein and modifications in assembly, materials, size, shape, and use.

From the above description of the invention, those skilled in the art will perceive improvements, changes and modifications. Such improvements, changes and modifications within the skill of the art are intended to be covered by the appended claims.

Having described the invention, the following is claimed:

1. A guide tube for use with a medical endoscope, said guide tube comprising:
an elongated tubular member having an inner annular membrane, an outer annular membrane, a proximal end portion, and a distal end portion opposite said proximal end portion, said inner and outer annular membranes defining a longitudinal axis extending between said proximal end portion and said distal end portion;
a lubricating fluid disposed between said inner annular membrane and said outer annular membrane, said lubricating fluid being disposed between said distal end portion and said proximal end portion, said inner annular membrane moving in a first axial direction along the medical endoscope as the medical endoscope is moved through a patient, said outer annular membrane correspondingly moving axially, but in a second, opposite axial direction, along the medical endoscope as the medical endoscope is moved through the patient.

2. The guide tube as set forth in claim 1 wherein said medical endoscope is a colonoscope.

3. The guide tube as set forth in claim 1 wherein said lubricating fluid comprises a saline solution.

4. The guide tube as set forth in claim 1 wherein said elongated tubular member moves at a rate if insertion that is one-half a rate of insertion of the medical endoscope.

5. The guide tube as set forth in claim 1 wherein said inner annular membrane and said outer annular membrane are constructed of a single membrane folded over itself.

6. The guide tube as set forth in claim 1 wherein said inner membrane moving in said first direction and said outer membrane moving in said second direction creates a rolling action of said elongated tubular member relative top the medical endoscope.

7. The guide tube as set forth in claim 6 wherein said rolling action mitigates sliding between said inner annular membrane and the medical endoscope.

8. The guide tube as set forth in claim 1 wherein said elongated tubular member moves at a rate of retraction that is one-half a rate of retraction of the medical endoscope.

9. The guide tube as set forth in claim 1 wherein said elongated tubular member is constructed as a single piece of material.

10. The guide tube as set forth in claim 9 wherein a portion of said inner annular member of said elongated tubular member will move to become part of said outer annular member as the medical endoscope is moved through the patient.

11. A method for viewing the interior of a colon of a patient, said method comprising the steps of:
providing a lubricating fluid between an inner annular membrane and an outer annular membrane;
moving the inner annular membrane in a first axial direction along a medical endoscope as the medical endoscope is moved through a patient; and
moving the outer annular membrane correspondingly, but in a second, opposite axial direction, along the medical endoscope as the medical endoscope is moved through the patient.

12. The method as set forth in claim 1 further including the step of moving the inner and outer membranes at a rate if insertion that is one-half a rate of insertion of the medical endoscope.

13. The method as set forth in claim 1 further including the step of constructing the inner and outer membranes from a single membrane folded over itself.

14. The method as set forth in claim 1 wherein the steps of moving the inner membrane a first direction and the outer membrane in a second direction creates a rolling action.

15. The method as set forth in claim 1 further including the step of mitigating sliding between the inner annular membrane and the medical endoscope and also mitigating sliding between the outer annular membrane and the colon of the patient.

16. The method as set forth in claim 1 further including the step of moving the inner and outer annular membranes at a rate of retraction that is one-half a rate of retraction of the medical endoscope.

17. The method as set forth in claim 1 wherein a portion of the inner annular membrane moves to become a part of the outer annular membrane as the medical endoscope is moved through the patient.

18. The method as set forth in claim 1 wherein the inner annular membrane and the outer annular membrane form the lubricating fluid.
